(19) United States
(12) Patent Application Publication
(10) Pub. No.: US 2006/0286161 A1
(43) Pub. Date: Dec. 21, 2006

(54) Injectable liposomal depots for delivering active ingredients

(76) Inventors: Steffen Panzner, Halle (DE); Silke Lutz, Halle (DE)

Correspondence Address: NORRIS, MCLAUGHLIN & MARCUS, P.A., 875 THIRD AVE, 18TH FLOOR, NEW YORK, NY, 10022, US

(21) Appl. No.: 10/556,123

(22) PCT Filed: May 8, 2004
(86) PCT No.: PCT/DE04/00998

§ 371 Date: Nov. 9, 2005

(30) Foreign Application Priority Data
May 9, 2003 (DE) 103 21 263.9
Feb. 4, 2004 (DE) 10 2004 005 783.4
Feb. 4, 2004 (DE) 10 2004 005 784.2
Mar. 4, 2004 (DE) 10 2004 010 720.3

(51) Int. Cl. A61K 48/00; A61K 31/573; A61K 9/127

(52) U.S. Cl. 424/450; 514/44; 514/171; 977/907

(57) ABSTRACT

The invention relates to liposomal formulations for the production of an injectable depot of peptide, protein and oligonucleotide active substances for sustained release and effect in a mammal body.

The invention relates to a liposomal delivery system for the delayed release of active substances and to the use of said system in basic research and clinics.

Following application, peptide and protein active substances undergo very rapid degradation in the body or elimination and therefore must be administered by repeated injections. To increase the “patient compliance”, a suitable delivery system is required which protects the active substance from degradation in the body, gradually releasing it into the bloodstream. Depot systems being injected subcutaneously or intramuscularly or implanted are used to this end. Liposomes are one possible form of such a carrier system. They are constituted of one or more lipid double layers that enclose in their inside an aqueous compartment allowing entrapment of water-soluble substances. The lipid double layer allows incorporation of lipophilic substances.

J. Controll. Rel. 64 (2000), 155-166, U.S. Pat. No. 5,766,627 and other papers by the authors present multivesicular aggregates of liposomes as injectable depot system for insulin, leuprolides and enkephalin, which are obtained by means of a double-emulsion process. Due to the addition of non-polar triglycerides, these multi-centered aggregates cannot be regarded as liposomes in a stricter sense because the triglycerides do not form any bilayer membranes and are not incorporated in the latter. Another drawback is that a water-immiscible oil phase is used in the production of said structures. Inclusion of larger proteins, in particular, will give rise to denaturation at the interface. Likewise, residues of organic solvents represent a regulatory problem that should not be underestimated.

According to the state of the art, liposomes composed of neutral, anionic or PEG lipids are used for depot systems, e.g. in WO 9920301 for a depot of γ-interferon, in Diabetes 31 (1982), 506-511, for a depot of insulin; furthermore, in Proc. Natl. Acad. Sci. 88 (1991), 10440-10444 for vaccination.

In BBA 1328 (1997), 261-272, various liposomal systems (unilamellar and multilamellar) of egg PC, egg PG, DPPC, DPPG, HP and cholesterol have been investigated for their reception in the lymphatic system and their biodistribution following subcutaneous administration. The review article Advanced Drug Delivery Reviews 50 (2001), 143-156, represents a continuation of the above investigations, demonstrating that liposomes smaller in size (<150 nm) migrate from a subcutaneous depot into the lymph.

According to the state of the art, neutral and negatively charged liposomes have been used in liposomal depot systems. For migration into the lymph to be absent, the liposomes must have a minimum size.

However, the production of large liposomes significantly greater than 150 nm is associated with technical and regulatory problems. More specifically, desirable sterile filtration of the particles subsequent to the production thereof is no longer possible.

Apart from peptides and proteins, oligonucleotides are likewise degraded very rapidly in the body by enzymes. In general, these active substances are administered at high doses by intravenous injections which, however, must be repeated frequently. For improved “patient compliance” and to allow reduction of the dose, a suitable delivery system is therefore required which protects the active substance against degradation in the body and effects slow and delayed liberation thereof.

Usually, delivery systems supporting the intracellular delivery of active substances following administration are in use today. These include liposomal systems, polymer-based systems (e.g. PEI) and viral carriers. Such intracellular strategies of delivery can result in dose reduction of the active substances. However, reduction of the injections cannot be achieved.

Another way of administering oligonucleotides involves depot systems being applied locally and liberating the active substances uniformly over a defined period of time. Such strategies of delivery do not necessarily support intracellular delivery of the active substances; rather, they result in a steady-state level of the active substance in blood or tissue for that period of time. In this way, the injection frequency can be reduced and, in addition, dose reduction is possible as a result of maintaining the concentration of active substance.

Micro- or nanoparticles made of biocompatible polymers represent one possible form of such a depot system. U.S. Pat. No. 6,555,525 describes the delayed release of antisense oligonucleotides from PLGA microcapsules following subcutaneous injection in a mouse leukemia model. Delayed release of oligonucleotides from PLGA-based micro- or nanocapsules has also been described in numerous other publications (for example, J. Drug Target. 5(4), 291-302, (1998); Gene Ther. 9 (23), 1607-16, (2002); Antisense Nucleic Acid Drug Dev. 9(5), 451-8, (1999); J. Control. Release 37, 173-183, (1995)).

Other polymer-based systems for the delivery of nucleic acids have been described in other printed documents. The authors of Methods: A Companion to Methods in Enzymology 18, 286-295, (1999), suggest e.g. the possible use of poly(hexyl cyanoacrylate) nanoparticles described therein as a depot system for oligonucleotides.

One drawback of micro- or nanoparticles made of polymers is the production process thereof. In most of such cases, emulsion processes must be employed, using organic water-immiscible solvents. These solvents must be completely removed after the end of the process. As a result, they represent a regulatory problem that should not be underestimated. Moreover, hydrolysis of the PLGA capsules gives rise to very low pH values inside the capsules, thus possibly impairing the integrity of the entrapped active substances. Thus, it is a well-known fact that purine bases are removed by hydrolysis from the nucleic acid backbone at low pH values.

Liposomes are another possible form of a carrier system for oligonucleotides. Numerous publications deal with the use of—mostly cationic—liposomal systems for the in vivo delivery of oligonucleotides (for example, Molecular Membrane Biology, 16, 129-140, (1999); BBA 1464, 251-261, (2000); Reviews in Biology and Biotechnology, 1(2), 27-33, (2001)). However, all these systems involve the common fact that the lipid mixtures used are constituted of unsaturated lipids such as DOTAP or DOPE and for this reason lack serum stability. As a result, such liposomes will rapidly release the enclosed active substance after injection. Also, complexes of preformed liposomes and nucleic acids (e.g. Lipoplexe) are frequently produced for the applications mentioned above. As a consequence of such complex formation, or of liposomal formulations mostly unstable in serum, stability of the oligonucleotides for a prolonged period of time, as required for a depot, cannot be guaranteed.

The object of the invention was therefore to provide new stable liposomal depot formulations for protein and peptide active substances and oligonucleotides, which would achieve long-term release of an active substance for at least one week and have good tolerability in an organism. Another object was to provide depot systems which avoid “burst release” of active substance or, if therapeutically indicated, achieve rapid initial partial release of active substance, followed by a sustained release of active substance.

The above technical object is accomplished by means of a depot system, particularly for delayed release of active substances, said system comprising liposomes (a) with saturated synthetic phosphatidyl cholines selected from the group of DMPC, DPPC and/or DSPC, (b) cholesterol with a percentage of from 35 to 50 mole-%, (c) cationic lipids selected from the group of DC-Chol, DAC-Chol, DMTAP, DPTAP and/or DOTAP with a percentage of from 5 to 20 mole-% in the liposomal membrane, and (d) a protein and/or peptide active substance, said formulation of active substances in liposomes being present in the form of aggregates when used as a depot. In addition to neutral lipids, such liposomes preferably comprise cationic lipids.

For example, positively charged liposomes undergo good aggregation with components of the serum or interstitial fluid, remaining at the puncture point in this condition. Advantageously, diffusion of the depot away from the puncture point is thus avoided.

The depots can be such in nature to either allow or prevent burst release. Depots with no burst release can be such that active substance adhering on the outside of the liposomes is detached and removed. Where burst release is advantageous, the active substance adhering on the outside of the liposomes will not be detached and removed.

Various methods of entrapping the—especially water-soluble—active substance in liposomes of the depot system are known to those skilled in the art. For inclusion of a desired active substance in liposomes, the active substance is dissolved in a buffer solution, for example, which is subsequently used to produce the liposomes. In the so-called passive inclusion, the relative volume enclosed by the liposomes being formed is an important issue. In passive inclusion, the inclusion efficiency is increased with increasing lipid concentration because the liquid volume enclosed by the lipid double layer is increased.

The teaching according to the present application has a number of advantages. Neutral/negatively charged liposomes, or micro- and nanoparticles of polymers are known to date, which have been used for the objects mentioned above.

The liposomes of the invention undergo aggregation with serum components and interstitial fluid components so that the depot remains at the site of puncture, thus preventing e.g. migration into the lymph. The lipid composition of the invention includes saturated backbone lipids providing integrity of the liposomes even in the aggregated state and thus improved protection of the active substance or longer depot times. The production process performs without organic, water-immiscible solvents possibly causing regulatory problems because complete removal thereof is difficult or damage to the active substance (proteins) may occur. There are no degradation products, as is the case with micro- and nanoparticles of polymers, which might do damage to the active substance (acid reaction during degradation of PLGA capsules). Depending on the requirements of therapy and on the active substance, variability is provided by the present/absent burst release character of the depot system.

In a preferred embodiment of the present invention, liposomes constituted of neutral and cationic lipids are used as liposomal depot system for the delayed release of therapeutic peptides and proteins of a wide variety of molar masses. J. Pharm. Sci. 89(3), 297-310, 2000, describes the absolute bioavailabilities of peptides and proteins of various size following subcutaneous application, wherein no significant reduction in bioavailability with increasing molar mass has been observed.

Therapeutic peptides and proteins undergo very rapid degradation in the body, for which reason they must be administered by repeated injections. The peptides and proteins, analogs thereof, related peptides, fragments, inhibitors and antagonists relevant to this embodiment of the invention comprise:

Transforming growth factors (TGF-alpha, TGF-beta), interleukins (e.g. IL-1, IL-2, IL-3), interferons (IFN-alpha, IFN-beta, IFN-gamma), calcitonin, insulin-like growth factors (IGF-1, IGF-2), parathyroid hormone, granulocyte colony-stimulating factor (GCSF), granulocyte macrophage colony-stimulating factor (GMCSF), macrophage colony-stimulating factor (MCSF), erythropoietin, insulins, amylins, glucagons, lipocortins, growth hormones, somatostatin, angiostatin, endostatin, octreotide, gonadotropin-releasing hormone (GNRH), luteinizing hormone-releasing hormone (LHRH), and effective agonists such as leuprolide acetate, buserelin, goserelin, triptorelin; platelet-derived growth factor; blood-clotting factors (e.g. factor VIII, factor IX), thromboplastin activators, tissue plasminogen activators, streptokinase, vasopressin, muramyl dipeptides (MDP), atrial natriuretic factor (ANF), calcitonin gene-related peptide (CGRP), bombesin, enkephalins, enfuvirtides, vasoactive intestinal peptide (VIP), epidermal growth factor (EGF), fibroblast growth factor (FGF), growth hormone-releasing hormone (GRH), bone morphogenetic proteins (BMP), antibodies and antibody fragments (e.g. scFv fragments, Fab fragments), peptide T and peptide T amides, herpes virus inhibitor, virus replication inhibition factor, antigens and antigen fragments, soluble CD4, ACTH and fragments, angiotensins, and ACE inhibitors, bradykinin (BK), hypercalcemia malignancy factor (PTH-like adenylate cyclase-stimulating protein), beta-casomorphins, chemotactic peptides and inhibitors, corticotropin-releasing factor (CRF), caerulein, cholecystokinins+fragments and analogs, galanin, gastric inhibitory polypeptide (GIP), gastrins, gastrin-releasing peptide (GRP), motilin, PHI peptides, PHM peptides, peptide YY, secretins, melanocyte-stimulating hormone (MSH), neuropeptide Y (NPY), neuromedins, neuropeptide K, neurotensins, phosphate acceptor peptide (c-AMP protein kinase substrates), oxytocins, substance P, TRH, as well as fragments, analogs and derivatives of the above substances.

Another preferred class of active substances for liposomal depots according to the invention are oligonucleotides. Oligonucleotides relevant to this embodiment of the invention are constituted of 5-100, preferably 5-40 and more preferably 10-25 nucleotides or base pairs. Moreover, the oligonucleotides can be present as a single strand (e.g. antisense oligonucleotides), double strand (e.g. small interfering RNA, decoy oligonucleotides), or in complex folding (e.g. aptamers, spiegelmers, ribozymes) . All oligonucleotides relevant to this invention are constituted of deoxyribonucleotides or ribonucleotides and chemically modified derivatives thereof (e.g. phosphorothioate DNA (PS), 2′-O-methyl-RNA (OMe), 2′-O-methoxyethyl-RNA (MOE), peptide nucleic acid (PNA), N3′-P5′-phosphoroamidate (NP), 2-fluroarabino nucleic acid (FANA), locked nucleic acid (LNA), morpholinophosphoroamidate (MF), cyclohexene nucleic acid (CeNA), tricyclo-DNA (tcDNA)). Moreover, copolymers and block copolymers of various nucleotides and so-called gapmers can be enclosed in the liposomes.

In one advantageous embodiment of the invention, aptamers or spiegelmers are enclosed in the liposomal depot. Aptamers are DNA- or RNA-based oligonucleotides with a complex three-dimensional structure. Owing to this structure, aptamers can bind to protein targets with high specificity and high affinity, thus having a therapeutic, mostly extracellular effect. Their functionality is virtually identical to that of monoclonal antibodies.

Unlike D-oligonucleotides, spiegelmers are constituted of L-ribose and L-2′-deoxyribose units. Just like aptamers, these mirror image nucleic acids specifically bind to protein targets. Owing to the chiral inversion, spiegelmers—in contrast to conventional D-oligonucleotides—have increased stability with respect to enzymatic degradation.

Furthermore, water-soluble active substances or water-soluble derivatives of active substances from the following classes of active substances are relevant to this invention: antibiotics (e.g. rifamycin SV Na salt, rifampicin, tetracyclin hydrochloride, kanamycin, penicillin G, ampicillin, novobiocin), antimycotic agents (e.g. amphotericin B, flucytosine), cytostatic agents (e.g. doxorubicin, daunorubicin, vincristin, cytarabin) , glucocorticoids (dexamethasone, prednisolone, hydrocortisone, betamethasone).

In addition to the above-mentioned classes of active substances, carbohydrates such as heparin or hyaluronic acid can be active substance molecules relevant to this invention. Membrane proteins, being difficult to introduce in the inner space of liposomes, do not represent preferred active substances in the meaning of the invention.

Membrane-forming and membranous lipids are possible as liposome-forming agents, and they can be of natural or synthetic origin. More specifically, these include cholesterol and derivatives, phosphatidyl cholines, phosphatidyl ethanolamines as neutral lipids. In a particularly preferred fashion, completely saturated compounds from this class are used, such as dimyristoyl, dipalmitoyl or distearoyl derivatives of phosphatidyl cholines (DMPC, DPPC, DSPC) and phosphatidyl ethanolamines.

For example, cationic lipids used in the practice of the invention comprise:
DAC-Chol 3-β-[N-(N′,N′-dimethylaminoethane)carbamoyl]-cholesterol
DC-Chol 3-β-[N-(N′,N′-dimethylaminoethane)carbamoyl]-cholesterol,
TC-Chol 3-β-[N-(N′,N′,N′-trimethylaminoethane)carbamoyl]cholesterol,
BGSC Bis-guanidinium-spermidine-cholesterol,
BGTC Bis-guanidinium-tren-cholesterol,
DOTAP (1,2-dioleoyloxypropyl)-N,N,N-trimethylammonium chloride,
DOSPER (1,3-dioleoyloxy-2-(6-carboxyspermyl)propylamide),
DOTMA (1,2-dioleyloxypropyl)-N,N,N-trimethylammonium chloride (Lipofectin®),
DORIE (1,2-dioleyloxypropyl)-3-dimethylhydroxyethylammonium bromide,
DOSC (1,2-dioleoyl-3-succinyl-sn-glycero choline ester),
DOGSDSO (1,2-dioleoyl-sn-glycero-3-succinyl-2-hydroxyethyl disulfide ornithine),
DDAB dimethyldioctadecylammonium bromide,
DOGS ((C18)₂GlySper3⁺) N,N-dioctadecylamido-glycyl-spermine (Transfectam®),
   (C18)₂Gly⁺N,N-dioctadecylamidoglycine, DOEPC 1,2-dioleoyl-sn-glycero-3-ethylphosphocholine or other O-alkylphosphatidyl cholines or ethanolamines, 1,3-bis(1,2-bis-tetradecyloxy-propyl-3-dimethylethoxyammonium bromide)-propan-2-ol (Neophectin®), and the saturated derivatives with dimyristoyl, dipalmitoyl or distearoyl chains of all above-mentioned lipids with unsaturated fatty acid and/or fatty alcohol chains.

Preferred cationic lipids used in the practice of the invention comprise cholesteryl-3-β-N-(dimethylaminoethyl) carbamate (DC-Chol), 3-β-[N-(N,N′-dimethylaminoethane)carbamoyl]cholesterol (DAC-Chol), (N-[1-(2,3-dimyristoyloxy)propyl]-N,N,N-trimethylammonium salt (DMTAP), (N-[1-(2,3-dipalmitoyloxy)propyl]-N,N,N-trimethylammonium salt (DPTAP), (N-[1-(2,3-dioleoyloxy)propyl]-N,N,N-trimethylammonium salt (DOTAP).

In a particularly preferred composition, saturated synthetic phosphatidyl cholines such as DMPC, DPPC or DSPC, cholesterol, the cationic lipids DC-Chol, DAC-Chol, DMTAP, DPTAP or DOTAP are used, and in a particularly preferred fashion the proportion of cationic lipids is between 5 and 20 mole-% and that of cholesterol between 35 and 50%.

In another advantageous embodiment of the invention, pH-sensitively cationic lipids are used, as disclosed in WO 02/066490 and U.S. Pat. No. 5,965,434 in an exemplary fashion. Liposomes containing such lipids can be imparted with a state of neutral charge by changing the pH, allowing easy removal of externally adhering active substance during the production process. Examples of pH-sensitively cationic compounds are:
histaminylcholesterol hemisuccinate (His-Chol), morpholine-N-ethylaminocholesterol hemisuccinate (Mo-Chol), 4-(2,3-bis-palmitoyloxy-propyl)-1-methyl-1H-imidazole (DPIM), cholesterol-(3-imidazol-1-ylpropyl) carbamate (CHIM).

The size of the liposomes according to the invention varies from 20 to 1000 nm, preferably from 50 to 800 nm, and more preferably from 50 to 300 nm.

Methods established in the prior art, such as extrusion through polycarbonate membranes, ethanol injection or high pressure homogenization, are used to produce the liposomes.

Passive inclusion is preferably used in those cases where large amounts of a readily soluble active substance are to be entrapped. To this end, liposomes with a lipid concentration of from 30 to 150 mM, preferably with a lipid concentration of from 50 to 120 mM, and more preferably with a lipid concentration of from 80 to 110 mM are produced in the presence of dissolved active substance.

Another method of entrapping water-soluble active substances is the so-called “advanced loading” method described in WO 01/34115 A2 which hereby is incorporated in the disclosure of the present invention. This method enables high inclusion efficiency. It is preferably used in those cases where the active substance is to be enclosed in the liposomes in a preferably cost-saving manner. This method, which is based on the interaction between the active substance and membrane-forming substances, operates at low ionic strength and at a pH value where the active substance is present in a state of anionic charge so as to undergo reversible electrostatic interaction with the cationic liposomal membrane.

For many proteins or peptides, this is the case under physiological conditions, i.e., at a pH value between 7 and 8. The charge of the active substances at a given pH can be inferred from data bases, such as SWISS-PROT, or can be estimated using well-known algorithms.

In another embodiment of the invention the passive inclusion method is combined with the advanced loading process. In this procedure, the advanced loading process is performed using a lipid concentration of from 30 to 150 mM, preferably a lipid concentration of from 50 to 120 mM, and more preferably a lipid concentration of from 80 to 110 mM, in order to significantly increase the inclusion rates compared to the separate methods.

Following liposome preparation, active substance adhering on the outside of the liposomal membrane can be detached and removed from the surface of the liposomes. This step is of crucial importance to the properties of the liposomal depot. Detaching the active substance from the liposome surface and removing it from the liposome suspension affords depot formulations having virtually no or only minimal “burst release”. In particular, this property is of crucial importance in those cases where active substances are to be administered which may give rise to toxic reactions in the body even during a briefly high concentration of active substance, as is the case during initial arrival. One example for this is insulin, overdosage of which may give rise to live-threatening hypoglycemic conditions. Termination of the existing interaction can be effected e.g. by changing the pH value or increasing the ionic strength.

Final removal can be effected using methods well-known to those skilled in the art, such as centrifugation, ultrafiltration, dialysis, or other chromatographic methods, so that at least 90% of the active substance is entrapped in the liposome and less than 10%, preferably less than 5% of the active substance is outside the liposome.

In another embodiment of the invention the active substance adhering to the liposomal membrane is not detached from the membrane, i.e., the pH value or ionic strength remains unchanged. In particular, this embodiment finds use with active substances where initial arrival of the active substance is toxicologically safe, as is the case e.g. with leuprolide acetate or many antibodies.

All or part of the free active substance, but more than 5%, preferably more than 10%, remains in the liposome suspension, providing for rapid initial arrival of active substance in the blood.

Another advantage of this embodiment is that the suspension can be lyophilized because, having equal concentrations of active substance on both the inner and outer surface of the membrane, release of active substance entrapped inside is minimized during the lyophilization process.

Leuprolide acetate ([D-Leu⁶Pro⁹Des-Gly¹⁰]-LHRH ethylamide) is a synthetically produced agonist of LHRH (luteinizing hormone-releasing hormone) and finds clinical use especially in cases of prostate cancer, endometriosis and premature puberty to lower the androgen level in the serum. Continuous administration of leuprolide acetate initially results in an increase of the testosterone level which is subsequently lowered down to the castration level. The initial increase of testosterone is due to stimulation of the LHRH receptors in the hypophysis and a thus induced secretion of LH which in turn stimulates testosterone production in the testicles. Eventually, said initial stimulation by leuprolide acetate is followed by a desensitization of the receptors in the hypophysis, thereby inhibiting the secretion of LH, which results in a decrease of the testosterone level. In a particularly preferred embodiment of the invention, leuprolide acetate is used as active substance of a depot system according to the invention.

In another preferred embodiment of the invention, antigens or antigen fragments are used as active substances of an inventive depot system for vaccination. In another preferred embodiment, therapeutically useful insulins are employed as active substances in a delivery system according to the invention.

The liposomal formulations of the invention can be used to produce a drug. In a preparatory step the liposomal formulations are placed in a physiologically tolerable medium. The conditions of a physiologically tolerable medium are well-known to those skilled in the art, comprising e.g. a pH value of from 7.3 to 7.6, preferably from 7.4 to 7.5, a salt content corresponding to about 150 mM NaCl or an osmolarity of about 320 osm.

The liposomal formulations of the invention can be injected subcutaneously or intramuscularly as a depot medicinal form. Furthermore, they can also be applied locally or topically.

The invention also relates to a kit comprising the depot system according to the invention, optionally together with information concerning combining the contents of the kit. The kit can be used in basic research and medicine. For example, the information can also be a reference to an internet address where further information can be obtained. The information can be a treatment regimen for a disease or e.g. instructions of how to use the kit in research.

Without intending to be limiting, the invention will be explained in more detail with reference to the following examples.

### DESCRIPTION OF THE FIGURES

FIG. 1 Comparison of liposomal depot systems of Example 4 of the present invention with an injected control sample (K3) in an animal model.

FIG. 2 Comparison of liposomal depot systems with leuprolide acetate of Example 4 of the present invention with an injected control sample (P29) in an animal model (serum level of leuprolide acetate).

FIG. 3 Comparison of liposomal depot systems with leuprolide acetate of Example 4 of the present invention with an injected control sample (P29) in an animal model (serum level of testosterone).

FIG. 4 Liposomal depot system with leuprolide acetate of Example 7 of the present invention in an animal model (serum level of leuprolide acetate)

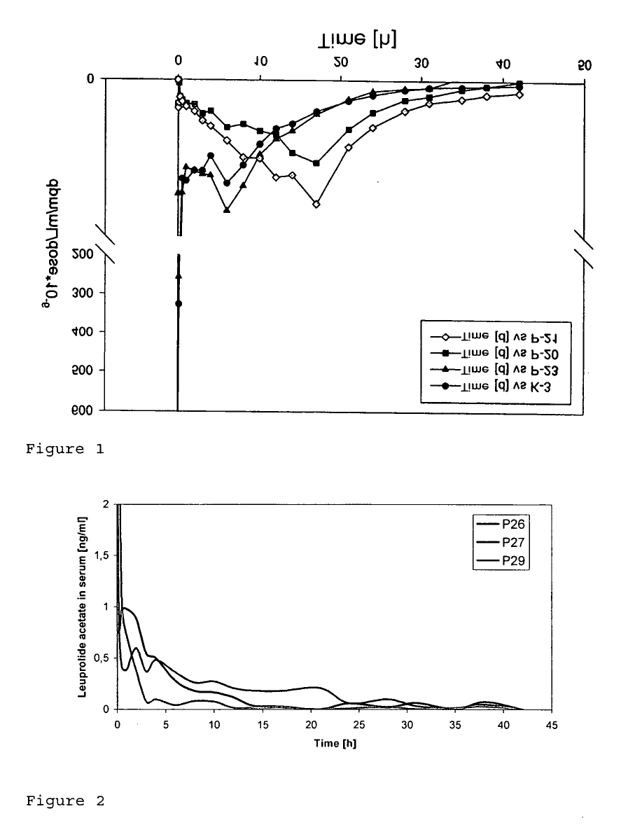

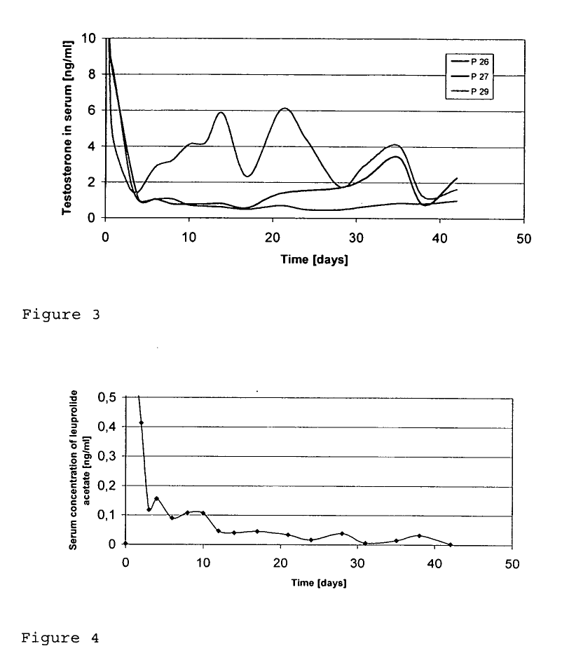

### EXAMPLES

### Example 1

### Inclusion of Insulin in Liposomes

Lipid mixtures having the following composition

| Formulation | Composition |
|---|---|
| I-1 | DPPC/DC-Chol/Chol 60:10:30 (mole-%) |
| I-2 | DPPC/DOTAP/Chol 50:10:40 (mole-%) |

are dissolved in chloroform at 50° C. and subsequently dried completely in vacuum in a rotary evaporator. The lipid film is added with human insulin solution (recombinant insulin; 4 mg/ml insulin in 10 mM HEPES, 300 mM sucrose, pH 7.5) in an amount so as to form a 50 mM suspension. Subsequently, this suspension is hydrated in a water bath at 50° C. for 45 minutes by agitating and treated in an ultrasonic bath for another 5 minutes. Thereafter, the suspension is frozen. This is followed by 3 cycles of freezing and thawing, each thawing being followed by a 5 minute treatment in the ultrasonic bath.

Following final thawing, the liposomes are subjected to multiple extrusions through a membrane having a pore width of 200 nm or 400 nm (Avestin LiposoFast, polycarbonate membrane with a pore width of 200 or 400 nm). Following extrusion, the resulting suspension is rebuffered by adding a stock solution of glycine-HCl, pH 3.5, and NaCl. After filtration of the liposomes through 0.8 μm, non-entrapped insulin is removed by triple sedimentation in an ultracentrifuge at 60,000×g, 45 min. A physiological pH is readjusted by adding a HEPES stock solution, pH 7.5. The amount of entrapped insulin is determined following extraction with CHCl₃ and CH₃OH, using RP-HPLC. Inclusion rates of 80-100% insulin are found.

### Example 2

### Inclusion of Alkaline Phosphatase (AP) in Liposomes

A lipid mixture having the following composition

| Formulation | Composition |
|---|---|
| AP-1 | DPPC/DOTAP/Chol 50:10:40 (mole-%) |

is dissolved in chloroform at 50° C. and subsequently dried completely in vacuum in a rotary evaporator. The lipid film is added with AP solution (from bovine intestinal mucosa) (5 mg/ml AP in 10 mM HEPES, 300 mM Sucrose, pH 7.5) in an amount so as to form a 50 mM suspension. Subsequently, this suspension is hydrated in a water bath at 50° C. for 45 minutes by agitating and treated in an ultrasonic bath for another 5 minutes. Thereafter, the suspension is frozen. This is followed by 3 cycles of freezing and thawing, each thawing being followed by a 5 minute treatment in the ultrasonic bath.

Following final thawing, the liposomes are subjected to multiple extrusions through a membrane having a pore width of 200 nm or 400 nm (Avestin LiposoFast, polycarbonate membrane with a pore width of 200 or 400 nm). Following extrusion, the ionic strength of the resulting suspension is increased by adding a stock solution of NaCl.

Removal of non-entrapped AP is effected by triple sedimentation in an ultracentrifuge at 60,000×g for 45 min.

Following organic precipitation with CHCl₃ and CH₃OH, the amount of entrapped AP is determined using a protein assay (BCA Protein Assay Reagent Kit, Perbio). In addition, the activity of entrapped AP is determined using an enzyme assay (p-nitrophenylphosphate test) . Inclusion rates of 40-50% AP are found.

### Example 3

### Inclusion of Inulin in Liposomes

Lipid mixtures having the following composition

| Formulation | Composition |
|---|---|
| P-20 | DPPC/DC-Chol/Chol 60:10:30 (mole-%) |
| P-21 | DPPC/DOTAP/Chol 50:10:40 (mole-%) |
| P-23 | DPPC/DPPG 40:60 (mole-%) |

are dissolved in chloroform at 50° C. and subsequently dried completely in vacuum in a rotary evaporator. The lipid film is added with ³H-inulin solution (18.5 MBq/ml ³H-inulin in 10 mM HEPES, 150 mM NaCl, pH 7.5) in an amount so as to form a 100 mM suspension. Subsequently, this suspension is hydrated in a water bath at 500C for 45 minutes by agitating. Thereafter, the suspension is frozen. This is followed by 3 additional cycles of freezing and thawing.

After the third thawing, the liposomes are subjected to multiple extrusions through a membrane having a pore width of 200 nm (Avestin LiposoFast, polycarbonate membrane with a pore width of 200). Removal of non-entrapped ³H-inulin is effected via gel filtration (G75 column Pharmacia). Following removal, the amount of entrapped ³H-inulin is determined in a scintillation counter. Inclusion rates of 10-25% ³H-inulin are found.

### Example 4

### Use of Liposomal Depot Systems in an Animal Model

The different liposomes of Example 3 were injected subcutaneously in healthy rats (3 animals per group) at a concentration of 20 mM lipid in a volume of 0.5 ml. A control sample with blank liposomes and non-encapsulated ³H-inulin was likewise administered subcutaneously in a volume of 0.5 ml. The pharmacokinetic data was obtained by blood sampling at varying points in time. The test period of the animal study was 6 weeks in total. The general condition of all animals was good over the test period. Only one animal in Group P20 showed heavy breath sounds for about 1 hour on test day 10.

The inulin content was determined by combustion of the blood samples (Oxidizer Ox 500, Zinser) and subsequent scintillation measurements.

The formulations and relative bioavailabilities up to t=42 d are illustrated in the following table:

| | | Relative |
|---|---|---|
| | | bioavailability |
| | | up to t = |
| Formulation | Composition | 42 days [%] |
| K-3 | DPPC/DPPG/Chol 50:10:40 (200 nm) + | 100 |
| | ³H-inulin outside | |
| P-20 | DPPC/DC Chol/Chol 60:10:30 (200 nm) | 136.5 |
| P-21 | DPPC/DOTAP/Chol 50:10:40 (200 nm) | 120 |
| P-23 | DPPC/DPPG 40:60 (200 nm) | 142 |

### Example 5

### Inclusion of Leuprolide Acetate in Liposomes

Lipid mixtures having the following composition

| Formulation | Composition |
|---|---|
| P-26 | DPPC/DC-Chol/Chol 60:10:30 (mole-%) |
| P-27 | DPPC/DOTAP/Chol 50:10:40 (mole-%) |
| L1 | DPPC/DC-Chol/Chol 60:10:30 (mole-%) (no removal) |

are dissolved in chloroform at 50° C. and subsequently dried completely in vacuum in a rotary evaporator. The lipid film is added with leuprolide acetate solution (95 mg/ml in 10 mM HEPES, 150 mM NaCl, pH 6, L1: 2.5 mg/ml) in an amount so as to form a 100 mM suspension. Subsequently, this suspension is hydrated in a water bath at 50° C. for 45 minutes by agitating. Thereafter, the suspension is frozen. This is followed by 3 additional cycles of freezing and thawing. Following final thawing, the liposomes are subjected to multiple extrusions through a membrane having a pore width of 400 nm (Avestin LiposoFast, polycarbonate membrane with a pore width of 400 nm). Removal of non-entrapped leuprolide acetate is effected by means of triple sedimentation in an ultracentrifuge at 60,000×g for 45 minutes (not with L1). The amount of entrapped leuprolide acetate is determined following extraction with CHCl₃ and CH₃OH, using RP-HPLC. Inclusion rates of about 15% leuprolide acetate are found.

### Example 6

### Use of Liposomal Depot Systems in an Animal Model

The different liposomes of Example 5 were injected subcutaneously in healthy male rats (3 animals per group) at a concentration of 25-30 mM lipid in a volume of 0.5 ml. A control sample with blank liposomes and non-encapsulated leuprolide acetate was likewise administered subcutaneously in a volume of 0.5 ml. The pharmacokinetic data was obtained by blood sampling at varying points in time, obtaining serum and determining the leuprolide acetate concentration in the serum by means of ELISA (Peninsula).

As leuprolide acetate influences the testosterone level of male rats, the testosterone concentration in the serum was also determined over the entire period using ELISA (DRG)

The test period of the animal study was 6 weeks in total. The general condition of all animals was good over the test period. The formulations and relative bioavailabilities up to t=42 d are illustrated in the following table:

| | | | Relative |
|---|---|---|---|
| | | | bioavailability |
| | | Size | up to t = |
| Formulation | Composition | [nm] | 42 days [%] |
| P-29 | DPPC/DC-Chol/Chol 60:10:30 + | 290 | 100 |
| | leuprolide, outside | | |
| P-26 | DPPC/DC-Chol/Chol 60:10:30 | 305 | 117 |

### Example 7

### Use of Liposomal Leuprolide Acetate in an Animal Model

Without removal of the active substance present outside, the liposomes of Example 5 were injected subcutaneously in healthy male rats (3 animals per group) in a volume of 0.5 ml. The leuprolide acetate dose was 2.5 mg per animal. The pharmacokinetic data was obtained by blood sampling at varying points in time, obtaining serum and determining the leuprolide acetate concentration in the serum by means of ELISA (Peninsula). The test period of the animal study was 6 weeks in total. The general condition of all animals was good over the test period. The formulation is shown in the following table:

| | | Dose |
|---|---|---|
| Formulation | Composition | [mg] |
| L1 | DPPC/DC-Chol/Chol 60:10:30 no removal | 2.5 |

### Example 8

### Inclusion of Cy5.5 Anti-CD40 ODN (Antisense Oligonucleotide) in Liposomes

A lipid mixture having the following composition:

| Formulation | Composition |
|---|---|
| AS1 | DPPC/DC-Chol/Chol 60:10:30 (mole-%) |

is dissolved in chloroform at 50° C. and subsequently dried completely in vacuum in a rotary evaporator. The lipid film is added with Cy5.5 anti-CD40 ODN (antisense oligonucleotide; 150 μg/ml in 10 mM HEPES, 300 mM Sucrose, pH 7.5) in an amount so as to form a 15 mM suspension. Subsequently, this suspension is hydrated in a water bath at 50° C. for 45 minutes by agitating and treated in an ultrasonic bath for another 5 minutes. Thereafter, the suspension is frozen. This is followed by 3 cycles of freezing and thawing, each thawing being followed by a 5 minute treatment in the ultrasonic bath.

Following final thawing, the liposomes are subjected to multiple extrusions through a membrane having a pore width of 200 nm or 400 nm (Avestin LiposoFast, polycarbonate membrane with a pore width of 200 or 400 nm). Following extrusion, the ionic strength of the resulting suspension is increased by adding a stock solution of NaCl.

Following removal of free active substance by triple sedimentation in an ultracentrifuge at 60,000×g for 45 min, the amount of entrapped Cy5.5 anti-CD40 ODN (antisense oligonucleotide) is determined using fluorescence spectroscopy.

The inclusion efficiency of the oligonucleotides is around 47%.

1. A depot system, for delayed release of active substances comprising liposomes having a membrane the liposomes comprising
saturated synthetic phosphatidyl cholines selected from one or more from the group consisting of DMPC, DPPC and DSPC,
cholesterol with a percentage ranging from 35 to 50 mole-%,
cationic lipids selected from the group of DC-Chol, DAC-Chol, DMTAP, DPTAP and DOTAP with a percentage ranging from 5 to 20 mole-% in the liposomal membrane, and
one or more selected from the group consisting of protein and peptide active substances.

2. The depot system according to claim 1, wherein the cationic lipids are cationic in a pH-sensitive fashion and selected from one or more from the group consisting of His-Chol and Mo-Chol.

3. The depot system according claim 1, wherein at least 90% of the active substance is enclosed in the liposome and less than 10% is outside the liposome.

4. The depot system according to claim 1, wherein the active substance is entrapped in the liposome and more than 10% thereof is outside the liposome.

5. The depot system according to claim 1, wherein the depot system is capable of sustaining the delivery of the active substance for at least 1 week.

6. The depot system according to claim 1, wherein the size of the liposomes varies from 20 to I ,000 nm.

7-18. (canceled)

19. The depot system according to claim 1, wherein the size of the liposomes varies from 50 to 800 nm.

20. The depot system according to claim 1, wherein the size of the liposomes varies from 50 to 300 nm.

21. The depot system according to claim 1, wherein the active substance comprises one or more from the group consisting of LHRH agonists and GnRH analogs.

22. The depot system according to claim 21, wherein the active substance comprises one or more from the group consisting of leuprolide, acetate, buserelin, goserelin and triptorelin.

23. The depot system according to claim 1, wherein the said active substance comprises insulin.

24. The depot system according to claim 1, wherein the said active substance comprises heparin.

25. The depot system according to claim 1, wherein said active substance comprises antigen fragments for vaccination.

26. The depot system according to claim 1, wherein the depot system is capable of a delayed release of active substance for at least one week and said active substance comprises oligonucleotides.

27. The depot system according to claim 26, wherein said oligonucleotides are constituted of 5-100 from the group consisting of deoxyribonucleotides, ribonucleotides and chemically modified derivatives thereof.

28. The depot system according to claim 26, wherein said oligonucleotides are constituted of 5-40 from the group consisting of deoxyribonucleotides, ribonucleotides and chemically modified derivatives thereof.

29. The depot system according to claim 26, wherein said oligonucleotides are constituted of 10-25 from the group consisting of deoxyribonucleotides, ribonucleotides and chemically modified derivatives thereof.

30. The depot system according to claim 26, wherein said oligonucleotides are present as one or more from the group consisting of a single strand, a double strand and in complex folding.

31. The depot system according to claim 30, wherein said oligonucleotides are present as a single strand, said single strand being present as antisense oligonucleotides.

32. The depot system according to claim 30, wherein said oligonucleotides are present as a double strand, said double strand being present as small interfering RNA, decoy oligonucleotides.

33. The depot system according to claim 30, wherein said oligonucleotides are present in complex folding as aptamers, spieglemers.

34. The depot system according to claim 1, wherein the depot system is capable of a delayed release of active substance for at least one week and said active substance comprises a water-soluble active substance derivative selected from one or more from the group consisting of active substances of antibiotic, antimycotic, cytostatic agents and glucocorticoids.

35. Method of administering the depot system according to claim 1, comprising the step of injecting the depot system subcutaneously or intramuscularly.

36. Method of administering the depot system according to claim 1, comprising the step of one or both from the group consisting of topical and local application to support healing processes.

37. A Drug comprising a depot system according to claim 1.
